(19) United States
(12) Patent Application Publication
(10) Pub. No.: US 2017/0011191 A1
(43) Pub. Date: Jan. 12, 2017

(54) PORTABLE DEVICE COMMUNICATING WITH PATIENT MONITORING DEVICE

(71) Applicant: GENERAL ELECTRIC COMPANY, Schenectady, NY (US)

(72) Inventors: Sureshkumar Rajasekar, Bangalore (IN); Nagesh Kumar Upadhyayula, Bangalore (IN)

(21) Appl. No.: 15/203,258

(22) Filed: Jul. 6, 2016

(30) Foreign Application Priority Data
Jul. 8, 2015 (IN) 3487/CHE/2015

(51) Int. Cl. G06F 19/00; A61B 5/00; A61B 5/0205

(52) U.S. Cl. CPC G06F 19/3418; A61B 5/02055; A61B 5/002; A61B 5/0022; A61B 5/743; A61B 5/486; A61B 5/746

(57) ABSTRACT

A portable device capable of communicating with a patient monitoring device is disclosed. The portable device comprises a processor configured to receive medical content presented by the patient monitoring device. The medical content is associated with physiological parameters of a patient monitored using the patient monitoring device. The processor emulates the medical content for presentation. A display of the portable device is configured to present the emulated medical content to the user. The portable device is also configured present guidance information received from the patient monitoring device. The user can access the guidance information for operating the patient monitoring device through the portable device.

### FIELD OF THE INVENTION

The subject matter disclosed herein relates to a patient monitoring system for monitoring physiological parameters of patients. More specifically it relates to a portable device capable of communicating with a patient monitoring system.

### BACKGROUND OF THE INVENTION

Patient monitoring systems help a user such as a physician or technician monitor and diagnose patients. Patient monitoring systems are generally configurable to monitor one or more different patient physiological parameters such as heart rate, blood pressure, body temperature, and saturation of peripheral oxygen (SpO2). Accordingly, a given patient monitoring system may comprise multiple monitoring subsystems such as an electrocardiograph, a blood pressure monitor, a thermometer, and/or a pulse oximeter. The user may not be always aware or knowledgeable of capabilities of the patient monitoring device in order to take correct actions or perform operations, especially in real time as the patient is being monitored. The user may be reluctant to modify or interact with the user interface presenting the monitored patient parameters to in order experiment or check any changes to be made in the parameters, any actions to be taken, or settings to be modified. Further the patient monitoring device can be configured in different configurations based on required care area. The user may not be able to quickly make these configuration changes unless the user is well-versed with the patient monitoring device. The user can use the help function available in these devices to make the configuration changes; however, the user may be reluctant to disturb the display showing the status of physiological parameters. Patient monitoring devices may have a demo mode that enables the user to understand the features of the device and how to operate the device. However, the demo mode is typically used when the patient monitoring device is not under clinical use.

When the user does not have instant guidance information for operating the patient monitoring device then it becomes difficult to operate in a clinical scenario. Thus there is a need for an improved way of providing guidance or help information to a user for operating a patient monitoring device.

### SUMMARY OF THE INVENTION

The object of the invention is to provide an improved system that provides a portable device that can communicate with a patient monitoring device to provide guidance information to a user for operating the patient monitoring device, which overcomes one or more drawbacks of the prior art. This is achieved by the system having the capability of as defined in the independent claim.

One advantage with the disclosed system is that it provides a portable device that emulates a user interface presented in the patient monitoring device. The user interface presents physiological parameters associated with a patient. The portable device also provides guidance information for performing operations and functions in the user interface based on user's need. The guidance information is presented to the user without disturbing the content i.e. status of the physiological parameters associated with the patient, presented in the user interface.

In an embodiment a portable device capable of communicating with a patient monitoring device is disclosed. The portable device comprises a processor configured to receive medical content presented by the patient monitoring device. The medical content is associated with physiological parameters of a patient monitored using the patient monitoring device. The processor emulates the medical content for presentation. A display of the portable device is configured to present the emulated medical content to the user.

In another embodiment a patient monitoring system is disclosed. The patient monitoring system comprises a patient monitoring device configured to monitor a plurality of physiological parameters associated with a patient and present on a patient monitoring device display medical content associated with the physiological parameter. A portable device is present that communicably connects to the patient monitoring device. The portable device comprises a processor configured to receive medical content presented in the patient monitoring device and emulate the medical content for presentation. The portable device also includes a display configured to present the emulated medical content to the user.

In yet another embodiment, a portable device capable of communicating with a patient monitoring device associated with a monitored patient is disclosed. The portable device comprises an emulation module configured to connect the portable device with the patient monitoring device, receive medical content presented by the patient monitoring device, and emulate the medical content for presentation; and a display configured to present the emulated medical content to a user.

A more complete understanding of the present invention, as well as further features and advantages thereof, will be obtained by reference to the following detailed description and drawings.

### BRIEF DESCRIPTION OF THE DRAWINGS

FIG. 1 is a schematic illustration of a portable device capable of communicating with a patient monitoring device according to an embodiment;

FIG. 2 illustrates the portable device communicating with the patient monitoring device over a network according to an exemplary embodiment;

FIG. 3 is a schematic illustration a portable device capable of communicating with a patient monitoring device according to an embodiment;

FIG. 4 is a schematic illustration of a portable device may be communicably connected to multiple patient monitoring devices according to an exemplary embodiment; and

FIG. 5 is a flow diagram of a method for establishing a communication between a portable device and a patient monitoring device for emulating medical content in the portable device according to an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical and other changes may be made without departing from the scope of the embodiments. The following detailed description is, therefore, not to be taken as limiting the scope of the invention.

As discussed in detail below, embodiments of a portable device capable of communicating with a patient monitoring device is disclosed. The portable device comprises a processor configured to receive medical content presented by the patient monitoring device. The medical content is associated with physiological parameters of a patient monitored using the patient monitoring device. The processor emulates the medical content for presentation. A display of the portable device is configured to present the emulated medical content to the user.

FIG. 1 is a schematic illustration of a portable device 100 capable of communicating with a patient monitoring device 102 according to an embodiment. A patient monitoring device 102 is used to monitor different physiological parameters of a patient. The physiological parameters may include, but are not limited to, temperature, cardiac electric activity, blood pressure and saturation of peripheral oxygen (SpO₂). The patient monitoring device 102 includes a central processing unit (CPU) or a processor 104, a display 106, a memory 108 and parameter sensor(s) 110. The processor 104 may receive data from the parameter sensor(s) 110 and process the data in a known manner. The processor 104 may transmit the processed data to the memory 108 for storage and/or the display 106 for communication to a user (i.e. a care giver, clinician, or doctor). In an embodiment the processor 104 may be configured to provide clinical decision support. Clinical decision support may involve any feedback or guidance adapted to facilitate patient diagnosis and/or treatment. The clinical decision support may also include trending formulations or representations, visualizations, predictive algorithms, protocols, recommendations, and guidance.

The parameter sensors 110 will hereinafter be described in a non-limiting manner as comprising specific exemplary devices. It should, however, be appreciated that the patient monitoring device 102 may alternately comprise any known monitoring devices. The parameter sensor 110 may comprise a digital thermometer configured to monitor a patient's body temperature and will hereinafter be referred to as the thermometer. The parameter sensor 110 may comprise an electrocardiogram (ECG) acquisition device adapted to measure a patient's cardiac electrical activity and will hereinafter be referred to as the ECG acquisition device. The parameter sensor 110 may comprise a non-invasive blood pressure (NIBP) measurement system adapted to monitor a patient's blood pressure and will hereinafter be referred to as the NIBP system. The parameter sensor 110 may comprise a pulse oximetry system adapted to measure SpO2 and will hereinafter be referred to as the SpO2 system.

Each physiological parameter may have associated alarms also in the patient monitoring device 102. For instance each physiological parameter may have a threshold value if crossed an associated alarm signal may be presented in the patient monitoring device 102 for the user's attention. The threshold values for each physiological parameter may be set by the user and accordingly these physiological parameters will be monitored. If the physiological parameter value deviates (exceeds or goes below) from the threshold value, then an associated alarm may be presented in the display 106 for the attention of the user. The threshold values for physiological parameters may be set differently for different patients. The monitored patient physiological parameters are displayed in the display 106 for the user to view and accordingly attend to the patient.

Alarms for the physiological parameters may be also displayed via the display 106 to alert the user whenever a physiological parameter value deviates from a corresponding preset threshold value or deviates from the corresponding threshold value at a certain degree. The alarm may be presented in different ways, for instance but not limited to: an auditory signal such as a beep, a visual signal such as an alert light on the display, or a combination thereof. Further the alarms may be also associated with any failure or potential failure in the patient monitoring device. For example, the alarm may be associated with a disconnected sensor, or a low power supply. It may be noted that the alarms may be presented to indicate any issues other than the examples explained hereinabove without deviating from the scope of this disclosure. The user of the portable device 100 may be remotely located or may not be in close proximity to the patient, so in accordance with an embodiment, alarms in the patient monitoring device 102 may be also presented on the portable device 100 to notify the user of the alarm.

The user (i.e. the caregiver, clinician, or doctor) may not be always near the patient and, hence, may not be able to view the patient monitoring device 102 and the display 106 at all times. The portable device 100 connected to the patient monitoring device 102 can present medical content presented in the display 106 of the patient monitoring device 106. The medical content will be hereinafter described in a non-limiting manner as comprising values or data, such as waveforms, associated with different monitored physiological parameters of the patient, alarms associated with the physiological parameters and so on. The medical content may also include different medical images that may be associated with the patient. Moreover the medical content may include different menu items and user interface (UI) elements that are needed for performing any operations such as but not limited to, selecting a particular configuration for operation, changing any physiological parameter that are monitored. In an embodiment, the portable device 100 emulates or duplicates the medical content presented in the display 106 in real-time. The portable device 100 includes a display 112 that presents the emulated medical content.

A processor 114 of the portable device 100 receives the medical content and processes it so that it can be presented in the display 112. The processing may involve rendering, compressing, or modifying the medical content to be presented in the display 112 of the portable device 110. The portable device 100 may be wired or wireles sly connected to the patient monitoring device 102. In an embodiment, the portable device 100 may be a handheld device carried by the user. The user can thus carry the portable device 100 along while moving and still view status of the patient physiological parameters. In case any alarms are presented due to deviation from any of the physiological parameters, then these alarms can also be presented in the display 112 in substantially real-time while presenting in the display 106. The alarms may be presented in the display 112 within 0-5 seconds. The user can view these alarms in the portable device 100 and can come to know of any emergency situation while not near to the patient.

The user may need assistance in navigating through the medical content in the patient monitoring device 102. The portable device 100 is capable of providing guidance information to the user for navigating through the medical content. For instance, the guidance information may be presented to enable the user to select UI element(s) for setting a particular configuration in the patient monitoring device 102. Different preset configurations may be set in the patient monitoring device 102. The configuration is set to enable the patient monitoring device 102 to function in a particular manner for monitoring one or more physiological parameters of the patient. In an embodiment, the guidance information may be presented as a manual describing how the user can access different UI elements to set a desired configuration. In another embodiment, the guidance information may be presented in the form of a media file that can be accessed or played by the user to understand how different UI elements can be accessed to set a desired configuration. The media file may include image files, video files and so on. It may be appreciated that there may be multiple embodiments wherein the guidance information can be presented to the user in different ways without deviating from the scope of this disclosure. Further it may be also noted that the guidance information may hereinafter described in different exemplary scenarios to include different data or information that assists the user. However it should be appreciated that the guidance information can include other information that assists the user for using the patient monitoring device within the scope of this disclosure.

In an embodiment, the patient monitoring device 102 may be capable of presenting trends of the health status associated with the patient. The trends may be part of the medical content presented through the display 106. These trends may be also emulated on the display 112 of the portable device 100. The trends may indicate the variation of health status of the patient for a defined period. For example the patient monitoring device 102 may present a trend of heart activity of the patient. The heart activity is indicated by ECG data for a defined period for instance past one week. This trend indicates the heart activity of the patient and accordingly required steps can be taken by the user. The trend of the ECG data is emulated in the display 112 in the portable device 100 held by the user. In an embodiment, the trend of the ECG data may be presented in the form of graphs. Alternatively, the ECG data trend may be presented in any other manner in different embodiments. The trend of the ECG data is an example and similarly trends associated with other health parameters may be also presented in the portable device 100.

The user may require some help or guidance for reviewing or obtaining the health status trend of the patient. The portable device 100 may present the guidance information for helping the user to review and obtain the health status trend. The guidance information may help the user to review and understand the health status trend presented. The guidance information may also enable the user to perform operations and access appropriate UI elements and steps involved for accessing the health status and configuring the patient monitoring device 102 to monitor the patient over a defined period to generate the health status trend. Further the operations may be associated with but not limited to configuring alarm(s) in response to a health status of the patient, monitoring a physiological parameter based on the health status of the patient and so on. All these operations can be performed on the display 112 of the portable device 100.

Considering an example, an alarm may need to be configured if blood pressure of the patient goes above a certain threshold value. This event of triggering an alarm in response to blood pressure variation above the threshold value may need to be configured in the patient monitoring device 102. The user can configure such an event in the patient monitoring device 102 using guidance information displayed on the display 106 of the portable device 100. The guidance information can act as a guide to the user for configuring the event using the portable device 100. The user is guided to access appropriate UI elements and steps for configuring the alarm settings.

In another example, the health status of patient may indicate a significant deviation of a physiological parameter from corresponding threshold value then the patient monitoring device 102 may be configured to automatically monitor another physiological parameter. The guidance information may also provide insight into how the user can configure the patient monitoring device 102 to automatically trigger monitoring the additional physiological parameter. The user may request the patient monitoring device 102 to present the guidance information through the portable device 100. In an embodiment the user may use a UI element on the display 106 associated with accessing the guidance information on the display 112. Alternatively, the display 112 may comprise a UI element that the user can select to access guidance information. The guidance information may be presented on the display 112, while the medical content, including physiological parameters and health status of the patient, may be continuously presented in the display 112. Without any disturbance to the medical content on display 112, the user can conveniently view and understand the guidance information for operating the patient monitoring device 102.

In an embodiment the guidance information may be stored in a memory 116 and retrieved when it is required. In this case the portable device 100 may not need to send any request to the patient monitoring device 102 for presenting the guidance information. Hence the user can access required guidance information locally and perform any operations in the portable device 100 so that same operations can be performed in the patient monitoring device 102.

The portable device 100 may connect with the patient monitoring device 102 using different communication techniques such as but not limited to, near field communication (NFC), Wi-Fi, bluetooth™ and zigbee™ and so on. The portable device 100 may be wired or wirelessly connected to the patient monitoring device 102. The patient monitoring device 102 may perform user authentication and/or device authentication for establishing a communication connection with the portable device 100. In an embodiment the user authentication can be performed by the user providing user identification details and/or a password. In another embodiment, the patient monitoring device 102 facilitates registering identification information of the user and the portable device 100. After the registration, any subsequent connection can be established when the portable device 100 comes in a communication range with the patient monitoring device 102. However it may be envisioned that different communication and authentication techniques are for establishing the communication between the portable device 100 and the patient monitoring device 102 in various embodiments.

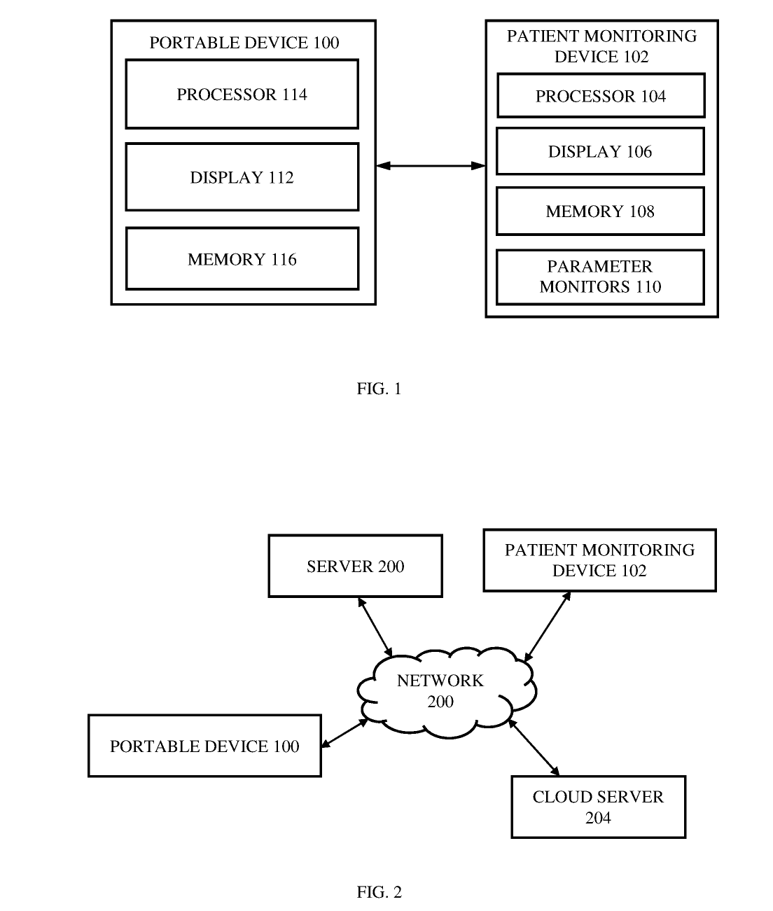

FIG. 2 illustrates the portable device 100 communicating with the patient monitoring device 102 over a network 200 according to an exemplary embodiment. The network 200 may include but is not limited to, a Local Area Network (LAN), a wireless LAN (WLAN), a Wireless Wide Area Network (Wireless WAN), a Wireless Personal Area Network (Wireless PAN), a Wireless Metropolitan Area Network (Wireless MAN), a Wireless Telecommunication Network, a 3^{rd} Generation communication (3G) network, a 4^{th} Generation communication (4G) network, public network (e.g. Internet), and a Long Term Evolution communication (4G LTE) network or any combination thereof. The portable device 100 may be held by the user located in a remote location with respect to the patient monitoring device 102 connected to the patient. In this embodiment the patient monitoring device 102 sends the medical content over the network 200 to reach the portable device 100. The portable device 100 emulates and presents the medical content presented in the patient monitoring device 102 through its display to the user in substantially real-time. For the purposes herein, substantially real-time shall be defined as within 0-5 seconds. The operations performed by the user by accessing UI elements on the display of the portable device 100 correspond to functions that are actually performed in the patient monitoring device 102. Further the patient monitoring device 102 is configured to transmit the guidance information to the portable device 100. The user can access the guidance information that helps the user to operate the patient monitoring device 102 through the portable device 100.

The portable device 100 is also capable of communicating with a server 202 over the network 200. When the portable device 100 receives request from the user for the guidance information, the server 202 may be contacted. The server 202 transfers the guidance information to the portable device 100 over the network 200 based on user's request. In one scenario the guidance information is downloaded and presented through the display of the portable device 100 to the user. The guidance information is downloaded in substantially real-time and may not be stored in the portable device 100. The server 202 also transfers the medical content to the portable device 100. In an embodiment, the medical content presented in the patient monitoring device 102 may be also available in the server 202 and the same medical content may be transferred to the portable device 100 in substantially real-time. The medical content is transferred from the server 202 upon receiving the user's request from the portable device 100.

In another embodiment the medical content and the guidance information may be transferred to the portable device 100 from a cloud server 204 over the network 200. Here the network 200 may be a cloud based network. The user's request may be processed by the cloud server 204. If the user's request is for obtaining the medical content, then the portable device 100 can download the medical content from the cloud server 204. The downloaded medical content is an emulation of the medical content presented in the display of the patient monitoring system 102. The medical content is downloaded in substantially real-time based on the user's request. Now in case the user's request is for obtaining the guidance information, the cloud server 204 processes this request to send the guidance information to the portable device 100. The cloud server 204 may be capable of storing different guidance information for operating the patient monitoring device 102. The cloud server 204 is explained as a single server processing the requests from the user, however it may be envisioned that the cloud server 204 may include multiple servers that provide such a service to the user. The cloud server 204 may be provided as a service by a service provider. In an embodiment access to the cloud server 204 may be provided to the user via a payment-based methodology. Here the user may have to subscribe to the cloud based service by paying appropriate fees to download the medical content and the guidance information into the portable device 100. In yet another embodiment, the medical content and the guidance information may be provided as live-streaming in the portable device 100 from the cloud server 204. The medical content and the guidance information may not be stored in the portable device 100 but may be stored temporarily in a buffer and removed after presentation to the user.

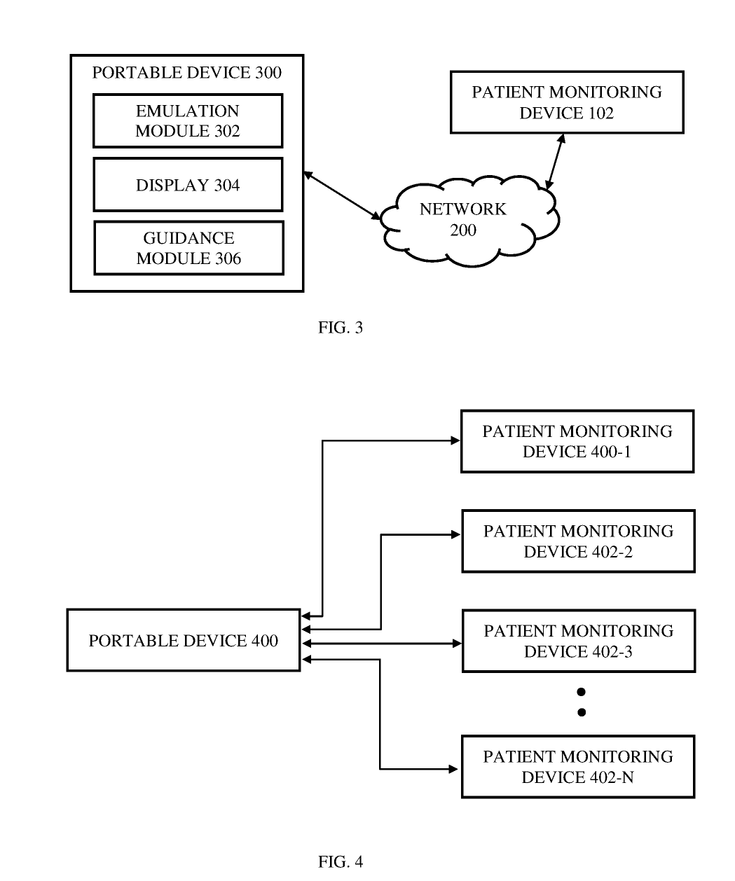

Now moving on to FIG. 3 a portable device 300 capable of communicating with a patient monitoring device 102 is shown according to an embodiment. The portable device 300 may be a handheld device such as a mobile device. The portable device 300 comprises an emulation module 302 capable of connecting the portable device 300 and the patient monitoring device 102. The portable device 300 may be wired or wirelessly connected to the patient monitoring device 102. The wireless connection may include but not limited to Wi-fi, bluetooth™, zigbee™ and so on. Thus the emulation module 302 may be configured to perform any user authentication and/or device authentication for establishing a communication connection between the portable device 300 and the patient monitoring device 102. In an embodiment the user authentication can be performed by the user providing user identification details and/or password. In another embodiment the emulation module 302 may facilitate in registering identification information of the user and the portable device 300 initially in the patient monitoring device 102. After the registration, any subsequent connection can be established when the portable device 300 comes in a communication range with the patient monitoring device. However it may be envisioned that various other embodiments may include different communication and authentication techniques or any combination thereof, for establishing a connection between the portable device 300 and the patient monitoring device 102.

The patient monitoring device 102 may be presenting the medical content as described in conjunction with FIGS. 1 and 2. The emulation module 302 may be configured to receive the medical content and emulate the content on a display 304. The medical content presented by the patient monitoring device 102 may be presented in substantially real-time in the portable device 300. A user request for emulating the medical content may be processed by the emulation module 302 and then the patient monitoring device 102 may be contacted to obtain the medical content. In an embodiment, the emulation module 302 may send a request to the patient monitoring device 302 to send the medical content. The patient monitoring device 302 transfers the medical content to the portable device 300 over a communication network. The communication network may be capable of transferring the medical content in real-time and hence may have higher bandwidth. The medical content received may be processed by the emulation module 302 and the presented to the user of the portable device 300.

The user handling the portable device 300 may be also capable of performing multiple operations in the patient monitoring device 300. The operations may include configuring patient monitoring device settings, selecting physiological parameters for monitoring, managing alarms, and performing functions associated with the medical content presented in the patient monitoring device 102 and so on. For performing these operations in the patient monitoring device 102 the user may require some guidance. A user input may be received at the portable device 300 that may be communicated to the patient monitoring device 102 for providing guidance information. A guidance module 306 communicates this user input to the patient monitoring device 102. The guidance information may be received by the guidance module 306 and presented to the user on the display 304. For instance the guidance information may be presented to enable the user to navigate through the medical content to select UI element(s) for setting a particular configuration in the patient monitoring device 102. Different preset configurations may be set in the patient monitoring device 102. The configuration is set to enable the patient monitoring device 102 to function in a particular manner for monitoring one or more physiological parameters of the patient. The guidance information enables the user to perform the operations in the patient monitoring device 102. The guidance information may be presented in different forms for instance it may be presented as a manual describing how the user can access different UI elements to set a desired configuration. In another embodiment the guidance information may be presented in the form of a media file that can be accessed or played by the user to understand how different UI elements can be accessed to set a desired configuration. The media file may include image files, video files and so on. In another embodiment, the guidance information may be UI elements embedded within the emulated medical content that once selected, provide Frequently Asked Questions (FAQs), or a menu to access to operations related to the medical content.

The emulation module 302 and the guidance module 306 may be part of an application configured in the portable device 300. In an embodiment the application can be downloaded and configured in the portable device 300. Thus the portable device 300 can be used by a user remotely located and can be connected to the patient monitoring device 102 for accessing the medical content and control the operations in the patient monitoring device 102. In another embodiment the portable device 300 may have the application built-in.

In a hospital environment multiple patient monitoring devices may be connected to a patient or to a plurality of different patients. As a result the user (i.e. the caregiver) may need to monitor different patients and their health status. The portable device used by the user may need to be connected to multiple patient monitoring devices. As illustrated in FIG. 4, a portable device 400 may be communicably connected to multiple patient monitoring devices such as a patient monitoring device 402-1, a patient monitoring device 402-2, a patient monitoring device 402-3 and patient monitoring device 402-n (hereinafter collectively referred to patient monitoring device 402-n) as according to an exemplary embodiment. The portable device 400 may need to be initially registered with each patient monitoring device. Then based on the requirement the user can connect the portable device 400 to appropriate patient monitoring device. The portable device 400 may connect with the patient monitoring device 402-1 at a particular instance. In an embodiment for establishing a connection the portable device 400 may need to send some connection information or authentication to the patient monitoring device 402-1. Once the connection is established the portable device 400 can start receiving the medical content presented by the patient monitoring device 402-1. The medical content includes the physiological parameters, health status, trending and their alarms associated with a patient monitored by the patient monitoring device 402-1. The user can also perform different operations on the patient monitoring device 402-1 using the portable device 400.

The user can disconnect the connection between the portable device 400 and the patient monitoring device 402-1 and establish a connection with the patient monitoring device 402-2. The process of establishing the connection with the patient monitoring device 402-2 may be similar. The user can view the medical content, control the operations and obtain the guidance information for performing the operations in the patient monitoring device 402-2. Similarly the portable device 400 can be connected to other patient monitoring devices as well. In order to connect with the patient monitoring device 402-1 again the portable device 400 may have to re-establish the connection. In an embodiment the portable devices 402-n may be connected to the patient monitoring devices and the same time. The user can shift between different patient monitoring devices based on their need and this case the portable device 400 may already connected to the patient monitoring devices 402-n. Here, the user may be provided an option to select the appropriate patient monitoring device in the portable device 400 and commence communication. The user can de-register any patient monitoring device from the portable device 400 at any time and communication can stop.

In another alternative embodiment the portable device 400 may be capable of presenting medical content from multiple patient monitoring devices at the same time. For instance the portable device 400 may be capable of presenting medical content from two patient monitoring devices in the single user interface on the display. More specifically the user interface may have two portions where each portion may be for presenting medical content from different patient monitoring devices. The user can perform operations in the patient monitoring devices by providing user input in appropriate corresponding portions of the user interface. Even though only few embodiments are described herein to explain how the user can visualize the medical content from different patient monitoring devices and control these devices, multiple other ways of accessing the medical content and operating the devices can be realized within the scope of this disclosure.

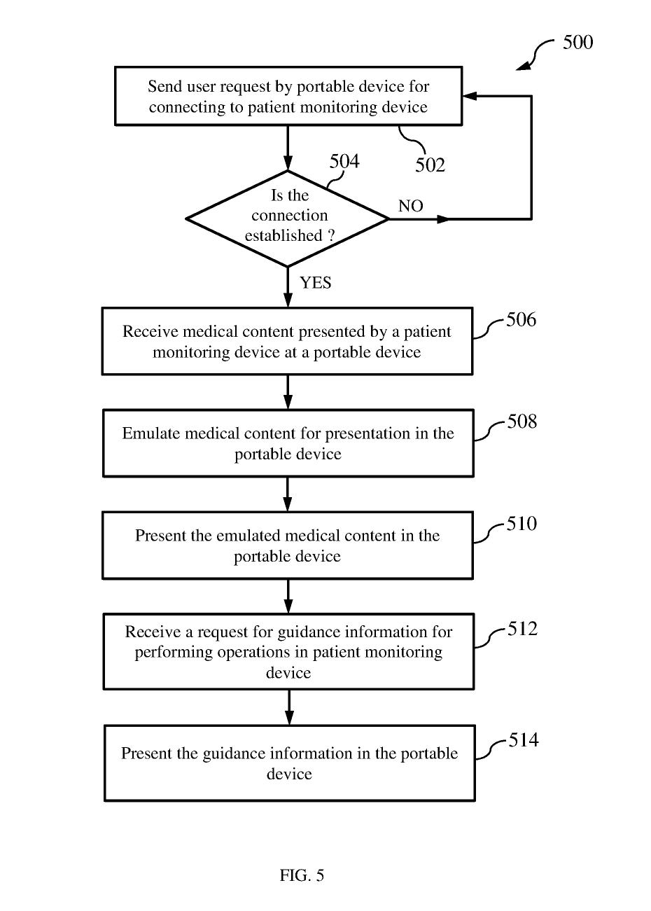

Turning now to FIG. 5, a method 500 for establishing a communication between a portable device and a patient monitoring device for emulating medical content in the portable device is shown according to an embodiment. It should be appreciated that in other embodiments, not all the steps of method 500 are required, and the steps may be performed in any order. A user using the portable device may send a user request for connecting to the patient monitoring device at step 502. The user request may be send along with some authentication information for authenticating the portable device at the patient monitoring device. The patient monitoring device may verify the authentication information and establish a connection. Before further communication between the portable device and the patient monitoring device a check is performed to confirm if a connection has been established at step 504. If the connection is not established, another user request may be send to the patient monitoring device by the portable device. Here the authentication information may be also resent to the patient monitoring device for establishing the connection. Then a check may be again performed at step 504 to confirm if the connection is established. If it is established then medical content presented in the patient monitoring device is transferred to the portable device based on user request. The portable device receives the medical content at step 506. The medical content is then emulated in the portable device at step 508. The emulated medical content is presented on a display of the portable device at step 510. The medical content presented in the patient monitoring device may be displayed in the portable device in substantially real-time for the user to visualize and analyze the health status of the patient.

The user may also request for guidance information that enables the user to perform different operations in the patient monitoring device at step 512. In an embodiment the guidance information may be stored in portable device. Alternatively the guidance information may be transferred from the patient monitoring device or a remote server capable of storing such information. Once the guidance information is received, then it is presented in the portable device at step 514.

The method 500 can be performed using a processor or any other processing device. The method steps can be implemented using coded instructions (e.g., computer readable instructions) stored on a tangible computer readable medium. The tangible computer readable medium may be for example a flash memory, a read-only memory (ROM), a random access memory (RAM), any other computer readable storage medium and any storage media. Although the method of diagnosing an ultrasound probe having a probe head comprising a plurality of transducer elements in accordance with another embodiment are explained with reference to the flow chart of FIG. 5, other methods of implementing the method can be employed. For example, the order of execution of each method steps may be changed, and/or some of the method steps described may be changed, eliminated, divide or combined. Further the method steps may be sequentially or simultaneously executed for diagnosing an ultrasound probe having a probe head comprising a plurality of transducer elements in accordance with another embodiment.

From the foregoing, it will be appreciated that the above disclosed is a portable device capable of communicating with a patient monitoring device. The portable device enables a user to monitor the health status of the patient from a remote location. Further the portable device also enables the user to control the operations in a patient monitoring device. The medical content presented in the portable device is in constant sync with respect to the medical content presented in the patient monitoring device. The user can have the medical content in substantially real-time and therefore the user located in another location can monitor the patient from a distance. The portable device also capable of providing guidance information for helping the user to perform operations in the patient monitoring device. The guidance information may be obtained at any time in response to a user request i.e. an on-demand availability of the guidance information. The guidance information is also updated frequently so that the user can have updated guidance for operating the patient monitoring device.

This written description uses examples to disclose the invention, including the best mode, and also to enable any person skilled in the art to practice the invention, including making and using any computing system or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A portable device capable of communicating with a patient monitoring device associated with a monitored patient, the portable device comprising:
a processor configured to:
receive medical content presented by the patient monitoring device;
emulate in substantially real-time the medical content for presentation; and
a display configured to present the emulated medical content to a.

2. (canceled)

3. The portable device of claim 1, wherein the medical content comprises at least one of a plurality of physiological parameters associated with the patient and an alarm.

4. The portable device of claim 1, wherein the processor is further configured to provide guidance information to guide the user in performing operations in the patient monitoring device.

5. The portable device of claim 4, wherein the operations comprise at least one of configuring patient monitoring device settings, selecting physiological parameters for monitoring, managing alarms, and performing functions associated with the medical content presented in the patient monitoring device.

6. The portable device of claim 4, wherein the processor is configured to receive the guidance information in response to user input.

7. The portable device of claim 4, wherein the processor is further configured to receive the guidance information from a server capable of communicating with the portable device in response to user's input.

8. (canceled)

9. The portable device of claim 4, wherein the processor is further configured to receive guidance information updates.

10. The portable device of claim 1, wherein the portable device is a handheld device.

11. The portable device of claim 1, wherein the portable device communicates with the patient monitoring device over at least one of a wireless and a wired connection.

12. A patient monitoring system comprising:
a patient monitoring device configured to
monitor a physiological parameter associated with a patient and present on a
patient monitoring device display medical content associated with the
physiological parameter; and
a portable device connected to the patient monitoring device, the portable device comprising:
a display; and
a processor configured to:
receive the medical content presented in the patient monitoring device display; and
emulate the medical content on the display,
wherein the medical content is received in substantially real-time.

13. (canceled)

14. The patient monitoring system of claim 12, wherein the medical content comprises at least one of a plurality of physiological parameters associated with the patient and an alarm.

15. The patient monitoring system of claim 14, wherein the processor is further configured to provide guidance information on the display to guide the user in performing operations in the patient monitoring device.

16. The patient monitoring system of claim 15, wherein the operations comprise at least one of configuring patient monitoring device settings, selecting physiological parameters for monitoring, managing alarms, and performing functions associated with the medical content presented in the patient monitoring device.

17. The patient monitoring system of claim 15, wherein the processor is configured to receive the guidance information in response to user input.

18. The patient monitoring system of claim 15, wherein the processor is further configured to receive the guidance information from a server capable of communicating with the portable device in response to user input.

19. (canceled)

20. The patient monitoring system of claim 14, wherein the processor is further configured to receive guidance information updates.

21. The patient monitoring system of claim 12, wherein the portable device communicates with the patient monitoring system over at least one of a wireless and a wired connection.

22. A portable device capable of communicating with a patient monitoring device associated with a monitored patient, the portable device comprising:
an emulation module configured to:
connect the portable device with the patient monitoring device;
receive medical content presented by the patient monitoring device;
emulate the medical content for presentation; and
a display configured to present the emulated medical content to a user.

23. The portable device of claim 23, wherein the medical content comprises at least one of a plurality of physiological parameters associated with the patient and an alarm.

24. The portable device of claim 23 further comprises a guidance module configured to provide guidance information for guiding the user to perform operations in the patient monitoring device.
